(19) United States
(12) Patent Application Publication
(10) Pub. No.: US 2013/0202712 A1
(43) Pub. Date: Aug. 8, 2013

(54) Compositions And Methods For Treating Or Preventing Immuno-Inflammatory Disease

(71) Applicants: Eric M. Ostertag, Lexington, KY (US); Paul C. Tumeh, Lexington, KY (US); P. Peter Ghoroghchian, Lexington, KY (US)

(72) Inventors: Eric M. Ostertag, Lexington, KY (US); Paul C. Tumeh, Lexington, KY (US); P. Peter Ghoroghchian, Lexington, KY (US)

(73) Assignee: VINDICO NANOBIO TECHNOLOGY, INC., Lexington, KY (US)

(21) Appl. No.: 13/582,736

(22) PCT Filed: Mar. 2, 2011
(86) PCT No.: PCT/US11/26863

§ 371 Date: Mar. 4, 2013

Related U.S. Application Data
(60) Provisional application No. 61/309,832, filed on Mar. 2, 2010.

(51) Int. Cl. A61K 9/127; A61K 31/7056; A61K 31/327; A61K 31/203; A61N 5/06; A61K 31/05

(52) U.S. Cl. CPC A61K 9/1273; A61N 5/0613; A61K 31/05; A61K 31/327; A61K 31/203; A61K 31/7056
USPC 424/497; 604/501; 514/733; 514/714; 514/559; 514/24

(57) ABSTRACT

The present invention relates to compositions and methods for the treatment of immuno-inflammatory conditions comprising the administration of a polyphenolic phytoalexin compartmentalized in a biocompatible and/or biodegradable polymeric carrier, and to the use of biocompatible and/or biodegradable polymeric carriers comprising resveratrol and block copolymers and these compositions with an additional compartmentalized pharmaceutically active agent.

### FIELD OF INVENTION

The present invention relates to compositions and methods for the treatment of immuno-inflammatory conditions comprising the administration of a polyphenolic phytoalexin compartmentalized in a biocompatible and/or biodegradable polymeric carrier (e.g. microparticle, solid nanoparticle, dendrimer, micelle or polymersome); to the use of biocompatible and/or biodegradable polymeric carriers comprising resveratrol and block copolymers and these compositions with an additional compartmentalized pharmaceutically active agent; and to methods of combination therapy comprising these compositions, and another separate therapy, which may include, for example, antibiotics, chemotherapeutic agents, Vitamin A and its derivatives, laser therapy, ultraviolet therapy, retinoic acid receptor and retinoid X receptor modulators, and benzoyl peroxide, which may be used for the treatment of acne.

### BACKGROUND OF THE INVENTION

Resveratrol or (3,5,4′-trihydroxystilbene) is a polyphenolic phytoalexin found in red wine and the peanut plant. Resveratrol has been studied to be useful in wound healing properties. In addition, it imitates the anti-aging effects observed in caloric-restricted mice fed a high fat diet by promoting insulin sensitivity, increasing mitochondrial cell content, and prolonging survival. Resveratrol has demonstrated antioxidant and anti-mutagen properties by inducing drug-metabolizing enzymes, inhibiting cyclooxygenase, and hydroperoxidase functions in mouse skin models. Unfortunately, resveratrol has very low solubility in water and low stability leading to significant challenges for dosage formulation and therapeutic applications. Hence, there is a need to develop formulations of resveratrol that are stable at higher concentrations and more water-soluble providing for enhanced therapeutic efficacy of poorly bioavailable agents.

One mechanism for overcoming previous formulation difficulties of resveratrol is to encapsulate it within a polymeric carrier (e.g. polymeric microparticle, solid nanoparticle, dendrimer, micelle, or polymersome). For example, polymersomes are a type of polymeric nanoparticle that is a vesicle or hallow-sac in aqueous solutions. Nanoparticles refer to structures whose sizes fall in the range of 1 nanometer (1 billionth of a meter) to several-hundred-nanometers in scale. Structures of this size take on novel properties and functions that are markedly different from those seen in their bulk constitutive materials. In particular, such small size, large surface-area-to-volume ratio, and multifunctionality offer intriguing opportunities for the utilization of nanoparticles as carriers in drug delivery and diagnostic imaging applications. Compared to their free drug formulations, nanoparticle-based agents generally have vastly improved biodistribution, pharmacokinetic, and toxicological parameters. In particular, nanoparticles facilitate the achievement of desirable biological responses while minimizing adverse side effects. The incorporation of resveratrol, with and without other pharmaceutical and immunomodulatory agents, within polymeric carriers, and specifically polymeric nanoparticles such as polymersomes, overcomes previous formulation challenges and enables its utilization for the treatment of acne vulgaris and other immuno-inflammatory diseases.

The best-known and most widely investigated nanoparticle platform is that of the liposome. Liposomes are non-toxic, non-antigenic, nanovesicles (“hollow sacks”) comprised of phospholipids, the building blocks of natural cell membranes. They have been utilized in a number of biotechnology applications to incorporate therapeutic and diagnostic agents within their water-filled cavities; as such, liposomes enable these drugs to be delivered more effectively while minimizing adverse systemic exposure. Nanovesicles exploit the enhanced permeability and retention (EPR) effect associated with leaky microvasculature to preferentially accumulate in diseased tissues. Randomized trials have demonstrated that liposome-based and free drug formulations have comparable response rates; the liposomal formulations, however, generally have decreased frequencies of drug-associated in vivo toxicities.

Despite these formulation and toxicologic advantages, the wide adoption of liposomes for drug delivery has encountered several challenges. In particular, liposomes lack many of the essential attributes of the ideal drug delivery vehicle:

1. Large Loading Capacity: liposomes have difficulty incorporating large amounts of molecular agents that do not easily dissolve in water.
2. Site-specific Targeting: current liposome formulations lack the ability to actively deliver their cargo specifically to only diseased cells; rather, they rely on a “passive targeting” mode dependent upon the leakiness of blood vessels near target sites.
3. Chemical and Mechanical Stability: the liposome's relatively fragile structure is easily compromised while being transported in the blood vasculature, resulting in premature loss of its cargo prior to reaching target cells.
4. Controlled Drug Release: liposomes require repeated dosing in order to maintain high therapeutic levels within the body over time.

Due to limitations in the intrinsic material properties of natural phospholipids, it has been extremely difficult to overcome these challenges. As a result, other nanometer-scale delivery vehicles have been developed. Most of these agents fall into several major classes: dendrimers, polymeric micelles, and solid nanoparticles (comprised of ceramics, polymers, proteins, etc) (see FIG. 1). While these alternative platforms address some of the deficiencies inherent to the liposome system, none can overcome all the aforementioned problems and, at the same time, also retain the liposome's unique advantages. Polymersome technology fulfills all of the above requirements of the ideal polymeric carrier for resveratrol.

There is a need to develop a mechanism by which high concentrations of polyphenolic phytoalexin or salts derived therefrom are administered to a subject for prevention or treatment of immunoinflammatory diseases which are highly bioavailable as compared to the bioavailability without polyphenolic phytoalexin.

### SUMMARY OF THE INVENTION

The invention relates to methods of manufacturing a medicament comprising a polymersome with non-immunogenic polymers using established FDA-approved building blocks. The invention relates to methods of treating or preventing an immuno-inflammatory disease comprising administering to a subject in need thereof a composition comprising a polymersome. The invention relates to methods of treating or preventing an immuno-inflammatory disease comprising administering to a subject in need thereof a composition comprising a polyphenolic phytoalexin. In some embodiments, the polymerosome comprises non-immunogenic polymers using established FDA-approved building blocks.

The invention also relates to polymer carriers comprising: (i) a plurality of copolymers; and (ii) at least one polyphenolic phytoalexin compound.

The invention also relates to pharmaceutical compositions comprising: a polymer carrier comprising: (i) a plurality of copolymers; and (ii) at least one polyphenolic phytoalexin or a salt derived from thereof. Hydrophilic (“water-loving”) polymers of poly(ethylene-oxide) and poly(ethylene-glycol) have been utilized to provide biocompatibility to the vesicles' surfaces and prolong the nanoparticles' blood circulation times, creating “stealth” delivery vehicles that can evade the body's natural defense mechanisms. In some embodiments the compositions, pharmaceutical compositions, and methods described herein comprise at least one or a combination of a plurality of hydrophobic (“water-fearing”) biocompatible polymers (e.g., poly(butadiene) and poly(ethyl-ethylene)) as well as biodegradable polymers (e.g. as poly(ε-caprolactone), poly(γ-methyl ε-caprolactone), poly (L-lactic acid), poly (D-lactic acid), and poly(lactic-co-glycolic acid)) have been utilized to constitute the vesicles' membrane portions. In some embodiments the compositions, pharmaceutical compositions, and methods described herein comprise at least one or a combination of a polymersomes degraded in physiological conditions, such as in the human body. In some embodiments the compositions, pharmaceutical compositions, and methods described herein comprise at least one or a combination of a plurality of other macroscale devices, poly(ε-caprolactone), poly(γ-methyl ε-caprolactone), poly (L-lactic acid), poly (D-lactic acid), and poly(lactic-co-glycolic acid). Poly(ε-caprolactone), poly(γ-methyl ε-caprolactone), poly (L-lactic acid), poly (D-lactic acid), and poly(lactic-co-glycolic acid) have been extensively utilized in medical applications, including as implantable biomaterials in drug delivery devices, bioresorbable sutures, adhesion barriers, and as scaffolds for injury repair via tissue engineering. In some embodiments the compositions, pharmaceutical compositions, and methods described herein comprise aliphatic esters that enable safe and complete biodegradation of the carrier and controlled release of their encapsulant payload. In some embodiments the compositions, pharmaceutical compositions, and methods described herein comprise polymersomes comprising poly(ethylene oxide)-block-poly(ε-caprolactone) or poly(ethylene oxide)-block-poly(γ-methyl ε-caprolactone). In some embodiments the compositions, pharmaceutical compositions, and methods described herein comprise polymerosomes with: 1) high permeability to small drug molecules; 2) maintenance of neutral pH environments upon degradation; 3) facility in forming blends with other polymers; and 4) suitability for long-term delivery afforded by slow erosion kinetics. In some embodiments the compositions, pharmaceutical compositions, and methods described herein comprise polymers that have safe and complete in vivo degradation as compared to the safety profiles and degradation profiles of polyphenolic phytoalexin without encapsulation.

Polymersomes exhibit several properties in common with liposomes, their lipid counterparts; for example, both classes of nanovesicles are capable of encapsulating hydrophilic compounds in their aqueous core. In some embodiments the compositions, pharmaceutical compositions, and methods described herein comprise polymerosomes with 1) high permeability to small drug molecules; 2) maintenance of neutral pH environments upon degradation; 3) facility in forming blends with other polymers; and/or 4) suitability for long-term delivery afforded by slow erosion kinetics. Utilization of these polymers has enabled the generation of nanocarriers that have safe and complete in vivo degradation. FIG. 2 is a representative schematic of the polymersome structure and self-assembly process.

Polymersomes exhibit several properties in common with liposomes, their lipid counterparts; for example, both classes of nanovesicles are capable of encapsulating hydrophilic compounds in their aqueous core. In some embodiments the compositions, pharmaceutical compositions, and methods described herein comprise polymerosomes with several distinguishing characteristics when compared to liposomes and other nanoparticle-based delivery vehicles:

1. Large Loading Capacity: depending upon the structure of their component copolymer blocks, polymersome membranes are significantly thicker (about 9 to about 22 nm) than those of liposomes (between about 3 to about 4 nm) thus enabling facile and stable loading of large pharmaceutical conjugates that possess poor water-solubility.
2. Robust Mechanical Stability: polymersomes possess enormous mechanical strength that makes them 5-50 times tougher than liposomes and substantially more stable than micellar structures constructed from similar molecular weight copolymers
3. Diverse Chemical Properties: by adhering to certain molecular geometric constraints, a number of composite materials can be used to form polymersomes and impart to these assemblies a rich diversity in chemical properties: including variable in vivo circulation times, site-specific biological adhesion, environmental responsiveness, as well as complete biodegradability
4. Self-assembly Process: polymersome production is economical and can be easily achieved in large-scale without the need for any costly post-manufacturing purification processes.

Polymersomes possess essentially all of the desirable attributes of liposomes while allowing for the additional incorporation of large amounts of therapeutic and molecular imaging agents, including ones that are not easily dissolved in water. In some embodiments the compositions, pharmaceutical compositions, and methods described herein comprise polymerosomes conjugated with biological active moieties, such as antibodies or peptides, to facilitate specific biological adhesion. Moreover, polymersomes are more stable, more economically feasible, and more chemically versatile than either phospholipid-based vesicles or other nanoparticle-based delivery vehicles. In some embodiments the compositions, pharmaceutical compositions, and methods described herein comprise polymerosomes co-incorporate both imaging and diagnostic agents to allow for direct tracking of the vesicles both in vitro and in vivo by non-invasive imaging. A comparison of polymersomes to other nanoparticle platforms is shown in FIG. 3.

In some embodiments, the invention relates to polymer carriers comprising biocompatible or biodegradable polymersomes comprised of poly(ethylene oxide) and poly(butadiene), poly(ε-caprolactone) or poly(γ-methyl ε-caprolactone) and incorporate resveratrol. In some embodiments the compositions, pharmaceutical compositions, and methods described herein comprise polymerosomes the poly(ethylene oxide) can have a number average molecular weight from about 1.3 to about 4.1 kD, from about 1.5 to about 3.8 kD, from about 1.7 to about 3.5 kD or from about 2.0 to about 3.0 kD. The block copolymer may have a fraction of poly(ethylene oxide) from about 8 to about 35 percent by weight, from about 10 to about 30 percent by weight, from about 15 to about 25 percent by weight, or from about 18 to about 22 percent by weight. In some embodiments the compositions, pharmaceutical compositions, and methods described herein comprise polymerosomes that comprise resveratrol segregated within the hydrophobic core of the polymersomes. In some embodiments the compositions, pharmaceutical compositions, and methods described herein comprise polymerosomes that comprise a polyphenolic phytoalexin or a salt derived therefrom segregated within the hydrophobic core of the polymersomes. In other embodiments, the polyphenolic phytoalexin incorporated within the aqueous interior of the polymersomes. In some embodiments the compositions, pharmaceutical compositions, and methods described herein comprise polymerosomes that comprise resveratrol incorporated within the aqueous interior of the polymersomes.

In some aspects, the invention concerns methods of treating or preventing an immuno-inflammatory disorder in an individual comprising administering to the individual an effective amount of a composition comprising a polyphenolic phytoalexin compound or derivative thereof or salt derived therefrom encapsulated within a polymeric carrier. In certain preferred embodiments, the polymeric carrier is a polymersome. In some embodiments, the polymersomes encapsulate a second pharmaceutically active agent (e.g. peptide, small molecule, polysaccharide, or nucleic acid) in addition to reserveratrol. In certain embodiments, the polymersomes incorporate an effective amount of a composition of at least one other anti-immunoinflammatory agent in addition to resveratrol and are used for the treatment of acne vulgaris. In another embodiment, the polymersomes are formed by a self-assembly process. In some embodiments, the treated individual is in need of such treatment or prevention.

There is a need to develop a mechanism by which high concentrations of polyphenolic phytoalexin are administered to a subject for prevention or treatment of immunoinflammatory diseases which are highly bioavailable as compared to the bioavailability without polyphenolic phytoalexin.

### BRIEF DESCRIPTION OF THE DRAWINGS

FIG. 1 demonstrates that nanoparticles can effectively incorporate a variety of different therapeutic and molecular imaging agents to increase their bioavailability, decrease their systemic toxicity, and enable more effective localization and delivery. A variety of nanocarriers have been constructed to date but only polymersomes may provide requirements of the ideal nanoparticle delivery platform.

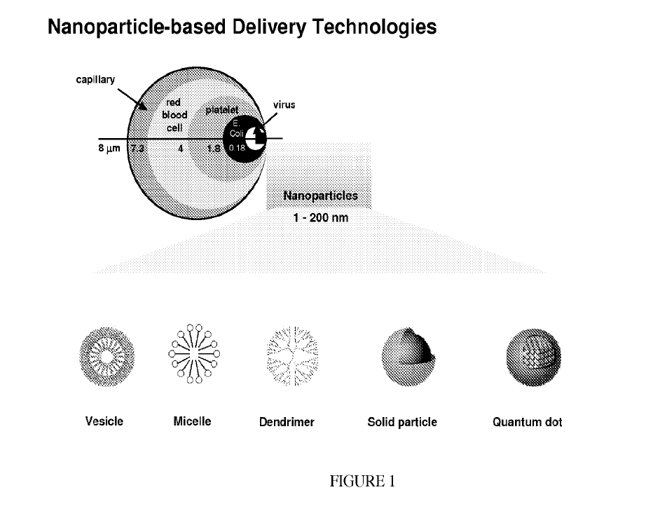

FIG. 2 is a representative schematic depicting the self-assembly of polymersomes. These polymer vesicles are spontaneous generated through a self-assembly process. Water-soluble molecules (green) are incorporated within the polymersome core while water-insoluble molecules (red) segregate in the vesicle membrane. Polymersome sizes can be controlled to yield a uniform distribution of nanoparticles.

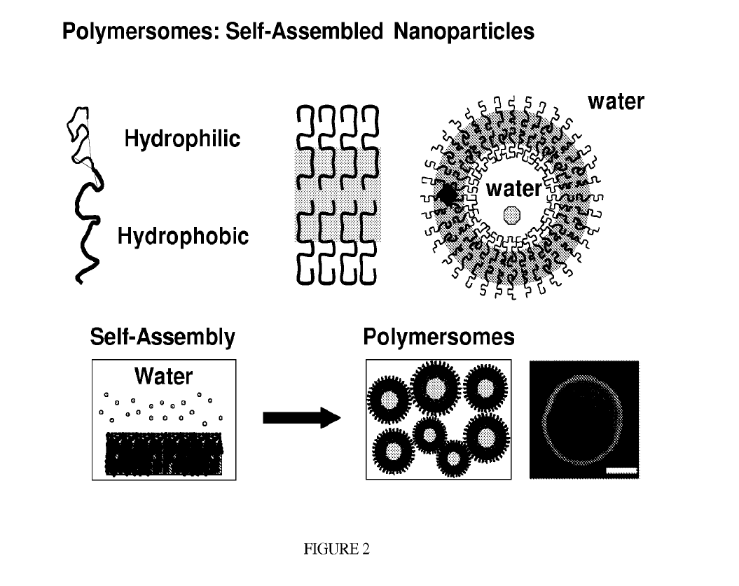

FIG. 3 shows a comparison of polymersomes to other nanoparticle-based carriers.

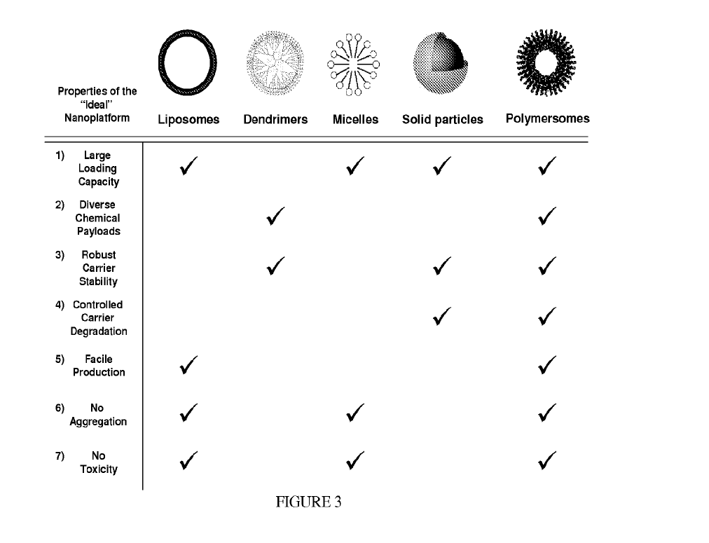

### DETAILED DESCRIPTION

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “salt” or refers to acidic salts formed with inorganic and/or organic acids, as well as basic salts formed with inorganic and/or organic bases. Examples of these acids and bases are well known to those of ordinary skill in the art. Such acid addition salts will normally be pharmaceutically acceptable although salts of non-pharmaceutically acceptable acids may be of utility in the preparation and purification of the polyphenolic phytoalexin or salt derived therefrom in question. Salts include those formed from hydrochloric, hydrobromic, sulphuric, phosphoric, citric, tartaric, lactic, pyruvic, acetic, succinic, fumaric, maleic, methanesulphonic and benzenesulphonic acids. In some embodiments, salts of the compositions comprising polyphenolic phytoalexin or salt derived therefrom may be formed by reacting the free base, or a salt, enantiomer or racemate thereof, with one or more equivalents of the appropriate acid. In some embodiments, pharmaceutical acceptable salts of the present invention refer to polyphenolic phytoalexin or salt derived therefrom having at least one basic group or at least one basic radical. In some embodiments, pharmaceutical acceptable salts of the present invention comprise a free amino group, a free guanidino group, a pyrazinyl radical, or a pyridyl radical that forms acid addition salts. In some embodiments of the invention, the pharmaceutical acceptable salts of the present invention refer to polyphenolic phytoalexin derivatives that are acid addition salts of the subject compounds with (for example) inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxybenzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. When several basic groups are present mono- or poly-acid addition salts may be formed. The reaction may be carried out in a solvent or medium in which the salt is insoluble or in a solvent in which the salt is soluble, for example, water, dioxane, ethanol, tetrahydrofuran or diethyl ether, or a mixture of solvents, which may be removed in vacuo or by freeze drying. The reaction may also be a metathetical process or it may be carried out on an ion exchange resin. In some embodiments, the salts may be those that are physiologically tolerated by a patient. Salts according to the present invention may be found in their anhydrous form or as in hydrated crystalline form (i.e., complexed or crystallized with one or more molecules of water). In some embodiments, the polyphenolic phytoalexin is a salt derived from 3,5,4′-trihydroxystilbene.

As used herein, the term “in vivo delivery” refers to delivery of a biologic by such routes of administration as topical, transdermal, suppository (rectal), pessary (vaginal), intravenous, oral, subcutaneous, intraperitoneal, intrathecal, intramuscular, intracranial, inhalational, oral, and the like.

As used herein, the term “immuno-inflammatory disease” refers to a variety of conditions, including but not necessarily limited to proliferative skin diseases, inflammatory dermatoses, autoimmune diseases, and certain cancers. Immunoinflammatory disorders result in the destruction of normal viable tissue by a combination of immune system dysregulation, inflammation, and abnormal cell division. Examples of immunoinflammatory disorders are acne vulgaris, acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-I diabetes; ulcerative colitis; uveitis; vasculitis; and Wegener's granulomatosis.

The term “subject” is used throughout the specification to describe an animal to whom treatment with the compositions according to the present invention is provided or administered. For treatment of those conditions which are specific for a specific subject, such as a human being, the term “patient” may be interchangeably used. In some instances in the description of the present invention, the term “patient” will refer to human patients. In some embodiments, the subject may be a mammal to whom the present invention is provided or administered. In some embodiments, the subject may be a non-human animal to whom the present invention is provided or administered. In some embodiments, the subject or patient will be in need of such treatment or will have been diagnosed as requiring such treatment.

The term “soluble” or “water soluble” refers to an aqueous solubility that is higher than 1/10,000 (mg/ml). The solubility of a substance, or solute, is the maximum mass of that substance that can be dissolved completely in a specified mass of the solvent, such as water. “Practically insoluble” or “insoluble,” on the other hand, refers to an aqueous solubility that is 1/10,000 (mg/ml) or less. Water soluble or soluble substances include, for example, polyethylene glycol.

The terms “treating” and “to treat”, mean to alleviate one or more symptoms, eliminate (all or partially) the causation either on a temporary or permanent basis, or to prevent or slow the appearance of symptoms. The term “treatment” includes alleviation, elimination of causation (temporary or permanent) of, or prevention of symptoms and disorders associated with any condition. The treatment may be a pre-treatment as well as a treatment at the onset of symptoms.

As used herein, the term “an effective amount” is meant the amount of a compound required to treat or prevent an immunoinflammatory disorder. The term “an effective amount” also refers to the amount of a compound, material, or composition, as described herein effective to achieve a particular biological result such as, but not limited to, biological results disclosed, described, or exemplified herein. Such results may include, but are not limited to, the effective reduction of symptoms associated with any of the disease states mentioned herein, as determined by any means suitable in the art. The effective amount of the composition may be dependent on any number of variables, including without limitation, the species, breed, size, height, weight, age, overall health of the subject, the type of formulation, the mode or manner or administration, the type and/or severity of the particular condition being treated, or the need to modulate the activity of the molecular pathway induced by association of the polyphenolic phytoalexin compound to its ligand or it naturally occurring biological target. Ultimately, the attending physician will decide the appropriate amount and dosage regimen.

As used herein, the term “nanometer” refers to a unit of measure of one-billionth of a meter.

As used herein, the term “pharmaceutically active agent” refers to any a protein, peptide, saccharide, nucleoside, inorganic compound, or organic compound that appreciably alters or affects the biological system to which it is introduced.

As used herein, the term “antibiotic” refers to any tetracycline, macrolide, fluoroquinolone, lincosamide, aminoglycoside, sulfonamide, sulfapyradine, chloramphenicol, vancomycin, metronidazole, or oxazolidinone.

As used herein, the term “Vitamin A and its derivatives” refers to retinol and related compounds that are either structurally derived or possesses functional analogy to Vitamin A.

As used herein, the term “laser therapy” refers to lasers that emit specific wavelengths within the UV (10-400 nm), visible (400-720 nm) and IR (720-1 000 000 nm) portions of the electromagnetic spectrum and include argon, frequency-doubled Nd:YAG/KTP, pulsed dye, ruby, alexandrite, diode, Nd:Yag, carbon dioxide, erbium:YAG.

As used herein, the term “ultraviolet therapy” encompasses irradiations with broadband UVB (290-320 nm), narrowband UVB (311-313 nm), 308 nm excimer laser, UVA1 (340-400 nm), UVA plus psoralens (PUVA), and extracorporeal photochemotherapy.

As used herein, the term “retinoic acid receptor and retinoid X receptor modulators” refers to any protein, peptide, saccharide, nucleoside, inorganic compound, organic compound that appreciably alters or affects the biological and biochemical pathways associated with nuclear retinoic acid receptors or retinoid X receptor.

As used herein, the term “vesicle” refers to semi-permeable bags of aqueous solution as surrounded (without edges) by a self-assembled, stable membrane composed predominantly, by mass, of either amphiphiles or super-amphiphiles. Thus, a biological cell would, in general, represent a naturally occurring vesicle. Smaller vesicles are also found within biological cells, and many of the structures within a cell are vesicular. The membrane of an internal vesicle serves the same purpose as the plasma membrane, i.e., to maintain a difference in composition and an osmotic balance between the interior of the vesicle and the exterior. Many additional functions of cell membranes, such as in providing a two-dimensional scaffold for energy conversion can be added to compartmentalization roles. For an intracellular vesicle, the environment outside the vesicle is the cytoplasm.

The term “drug delivery” refers to the method or process of administering a pharmaceutical compound such as the active agents described herein, to achieve a therapeutic effect in humans or any other animal.

The term “drug delivery vehicles” are agents with no inherent therapeutic benefit but when combined with a pharmaceutical agent for the purposes of drug delivery result in modification of the pharmaceutical compounds solution concentration, bioavailability, absorption, distribution and elimination for the benefit of improving product efficacy and safety, as well as patient convenience and compliance. Most common drug delivery vehicles include the preferred non-invasive peroral (through the mouth), topical (skin), transmucosal (nasal, buccal/sublingual, vaginal, ocular and rectal) and inhalation routes. Many medications such as peptide and protein, antibody, vaccine and gene based drugs, in general may not be delivered using these routes because they might be susceptible to enzymatic degradation or can not be absorbed into the systemic circulation efficiently due to molecular size and charge issues to be therapeutically effective. For this reason many protein and peptide drugs have to be delivered by injection. For example, many immunizations are based on the delivery of protein drugs and are often done by injection. Current efforts in the development of drug delivery vehicles include the generation of agents that enable targeted delivery in which the drug is only active in the target area of the body (for example, in cancerous tissues) and sustained release formulations in which the drug is released over a period of time in a controlled manner from a formulation. In some embodiments, drug delivery vehicles provide targeted delivery or sustained release formulations can be comprised of a number of materials including lipids, peptides, proteins, polymers, polysaccharides, nucleic acids, or small molecules.

As used herein, the term “polymer carrier” refers a class of drug delivery vehicles were a chemical polymer (i.e. multimer of similar individual units that are chemically attached to one another but provide novel properties from the individual consistituent components) are utilized for drug delivery. Examples of polymeric carriers include single/multiple/branch chain polymers that are conjugated to the pharmaceutical agent of interests, solid microparticles, solid nanoparticles, micelles, dendrimers, and vesicles/polymersomes. In some embodiments, the polymer carrier is a polymersome. In some embodiments, the polymer carrier is a microparticle, nanoparticle, micelle, polymerosome, or vesicle. In some embodiments, the polymer carrier is a microparticle. In some embodiments, the polymer carrier is a nanoparticle. In some embodiments, the polymer carrier is a micelle. In some embodiments, the polymer carrier is a vesicle. In some embodiments of the invention, polymersomes of the invention are vesicles which are assembled from synthetic multi-block polymers in aqueous solutions and does not include lipids or phospholipids as its majority component. In some embodiments of the invention, the polymersomes assemble during processes of lamellar swelling, e.g., by film or bulk rehydration or through an additional phoresis step or by other known methods. In some embodiments of the invention, polymersomes form by “self assembly,” a spontaneous, entropy-driven process of preparing a closed semi-permeable membrane.

As used herein, the term “copolymer” is a polymer derived from two (or more) monomeric species, as opposed to a homopolymer where only one monomer is used. Copolymerization refers to methods used to chemically synthesize a copolymer. In some embodiments of the invention, the copolymer comprise alternating copolymers with regular alternating A and B units In some embodiments of the invention, the copolymer comprises periodic copolymers with A and B units arranged in a repeating sequence (e.g. (A-B-A-B-B-A-A-A-A-B-B-B)n). In some embodiments of the invention, the copolymer comprises statistical copolymers are copolymers in which the sequence of monomer residues follows a statistical rule. If the probability of finding a given type monomer residue at a particular point in the chain is equal to the mole fraction of that monomer residue in the chain, then the polymer may be referred to as a truly random copolymer. In some embodiments of the invention, the copolymer comprises block copolymers that comprise two or more homopolymer subunits linked by covalent bonds. In some embodiments of the invention, the copolymer comprises a block polymer that further comprises an intermediate non-repeating subunit, known as a junction block. In some embodiments of the invention, the copolymer comprises a block polymer with two or three distinct blocks are called diblock copolymers and triblock copolymers, respectively. In some embodiments of the invention, the copolymer comprises an arrangement of branches in the polymer structure. Linear copolymers consist of a single main chain whereas branched copolymers consist of a single main chain with one or more polymeric side chains. In some embodiments, the copolymer comprises branched copolymers include star copolymers, brush copolymers, comb copolymers, or any combination of the above-mentioned embodiments. Polymer carriers and copolymers of the claimed invention may be made by any number of processes known in the art. In some embodiments, the copolymers and polymer carriers may be manufactured through methods disclosed in the following references, each incorporated by reference in its entirety: Bellas, Vasilios; Rehahn, Matthias (2007). “Universal Methodology for Block Copolymer Synthesis”, Macromolecular Rapid Communications 28: 1415. Bellas, Vasilios; Rehahn, Matthias (2009). “Block Copolymer Synthesis via Chemoselective Stepwise Coupling Reactions”, Macromolecular Chemistry and Physics 210: 320. In some embodiments, the copolymers and polymer carriers may be manufactured through methods triblocks, tetrablocks, multiblocks, etc. Diblock copolymers are made using living polymerization techniques, such as atom transfer free radical polymerization (ATRP), reversible addition fragmentation chain transfer (RAFT), ring-opening metathesis polymerization (ROMP), and living cationic or living anionic polymerizations. In some embodiments, the copolymers and polymer carriers may be manufactured through chain shuttling polymerization. The most powerful strategy to prepare block copolymers is the chemoselective stepwise coupling between polymeric precursors and heterofunctional linking agents. In some embodiments, the copolymer comprises tetrablock quarterpolymers ABCD.

In some embodiments of the invention, the compositions, pharmaceutical compositions, and methods of the invention comprise the manufacture, and use of graft copolymers. A used herein, the terms “graft copolymers” are a special type of branched copolymer in which the side chains are structurally distinct from the main chain. In some embodiments of the invention, the graft copolymers of the main chain and side chains are composed of distinct homopolymers. In some embodiments of the invention, the individual chains of a graft copolymer may be homopolymers or copolymers. Note that different copolymer sequencing is sufficient to define a structural difference, thus an A-B diblock copolymer with A-B alternating copolymer side chains is properly called a graft copolymer.

In some embodiments of the invention, the graft copolymers absorb energy when the substance is hit, so it is much less brittle than ordinary plastics. In some embodiments of the invention, the graft copolymers are high-impact copolymers.

The invention also relates to compositions or pharmaceutical composition comprising a polyphenolic phytoalexin or a salt derived therefrom for treating and/or preventing acne. In some embodiments, the pharmaceutical composition or composition comprises a polymeric carrier comprising a polyphenolic phytoalexin or a salt derived therefrom for treating and/or preventing acne. In some embodiments, the pharmaceutical composition or composition comprises a polymeric carrier comprising a polyphenolic phytoalexin or a salt derived therefrom for treating and/or preventing immuno-inflammatory cutaneous disease in a subject in need thereof. In some embodiments, the pharmaceutical composition or composition comprises a polymeric carrier comprising a polyphenolic phytoalexin or a salt derived therefrom useful for treating and/or preventing immuno-inflammatory cutaneous disease caused by pathogens in a subject in need thereof. In some embodiments, the pharmaceutical composition or composition comprises a polymerosome comprising a polyphenolic phytoalexin or a salt derived therefrom for treating and/or preventing immuno-inflammatory cutaneous disease caused by pathogens in a subject in need thereof. In some embodiments, the pharmaceutical composition or composition comprises a polymerosome comprising a anitmicorbial agent or a salt derived therefrom for treating and/or preventing immuno-inflammatory cutaneous disease caused by pathogens in a subject in need thereof. In some embodiments, the antimicrobial molecule is resveratrol. In some embodiments, the cutaneous pathogen is acne vulgaris.

The invention also relates to methods of manufacturing polymer carriers. While much research in dermatology has focused on liposomal and microsponge drug delivery systems, studies on more advanced nanotechnology systems have been relatively scarce and remain in a nascent investigatory stage. In some embodiments, the invention relates to methods of manufacturing plomer carriers comprising encapsulating anti-acne compounds into polymersomes, such as polymeric nanovesicles (PNV). In some embodiments, the polymer carriers may be manufactured in a stepwise manner, as evidenced by previous reports that demonstrate successful incorporation of similar size/amphiphilic compounds. In contrast to other drug delivery systems demonstrating immunological properties and in some embodiments, the PNV remains inert an non-immunogenic in the presence of human blood mononuclear cells. In some embodiments of the invention, incorporated compounds can be successfully released from the PNV in a time-dependent manner, thereby reducing toxicity to human mononuclear cells while demonstrating equivalent and/or enhanced bacterial cell kill.

While significant nanotechnology investigatory efforts have focused on intravenous drug delivery and in vitro diagnostics, the potential application of nanoscale synthetic soft materials as the next generation of cutaneous drug delivery systems remains largely unexplored. Skin has evolved over hundreds of thousands of years to serve as a protective barrier against environmental insults while also minimizing water loss. Therefore its natural low permeability to molecules has greatly challenged the penetration and efficacy of topical therapeutics.

The invention also relates to methods of treating or preventing an immuno-inflammatory disease or condition with a resveratrol-polymeric carrier formulation. Conventional acne medications are intrinsically irritating to human skin resulting in a significant need to improve the efficacy and side effect profile of these medications. Topical medications such as tretinoin and benzoyl peroxide have proven effective however their effective doses lead to significant skin irritation and decreased patient compliance. Specifically, benzoyl peroxide can cause dryness, and redness, while tretinoin is known to cause sun sensitivity, dryness, and peeling. Specific limitations associated with current acne treatments are effectively circumvented by using carriers such as liposomes and microsponge drug delivery systems to deliver the anti-acne medications in vivo. Advanced polymeric carriers described in this invention allow for high-dose incorporation, effective shelf-life storage, and controlled release of such compounds and maximize therapeutic effect while minimizing adverse side effects. Incorporation and release of resveratrol from polymeric nanocarriers demonstrates promise for the clinical treatment of acne and further does not lead to skin irritation, dryness, redness, sun-sensitivity, or peeling as is associated with currently available free-drug formulation used for acne treatment.

The invention also relates to methods of treating or preventing a disorder transiently responsive to resveratrol in a subject in need thereof but where patients experience limited therapeutic benefit due to drug delivery shortcomings. Specific embodiments of this invention enable the delivery of resveratrol intravascularly, intramuscularly, intranasally, intramucosally (e.g. vaginal, anal), inhalationally, and/or orally for disease treatment.

The invention also relates to methods of delivering a pharmaceutical composition comprising from about 0.01 mg to about 500 mg, from about 0.1 mg to about 500 mg, from about 1 mg to about 500 mg, from about 10 mg to about 250 mg, from about 50 mg to about 200 mg, from about 75 mg to about 150 mg of polyphenolic phytoalexin into a subject in need thereof by in vivo administration.

In some embodiments of the method, the pharmaceutical composition is administered orally, intranasally, vaginally, rectally, or transdermally. In some embodiments, at least one other therapeutic agent is co-administered with the pharmaceutical composition comprising from about 0.01 mg to about 500 mg, from about 0.1 mg to about 500 mg, from about 1 mg to about 500 mg, from about 10 mg to about 250 mg, from about 50 mg to about 200 mg, from about 75 mg to about 150 mg of polyphenolic phytoalexin.

In some embodiments the method includes administration of a pharmaceutical composition comprising a concentration of polyphenolic phytoalexin or salt derived therefrom is from about 0.01% to about 90% of the dry matter weight of the composition. In some embodiments, includes administration of a pharmaceutical composition comprising a polyphenolic phytoalexin or salt derived therefrom wherein the subject is administered a total amount of from about 1 mg to about 500 mg of polyphenolic phytoalexin or salt derived therefrom per day. In some embodiments, includes administration of a pharmaceutical composition comprising a polyphenolic phytoalexin or salt derived therefrom wherein the subject is administered a total amount of up to about 25 mg of polyphenolic phytoalexin or salt derived therefrom per day. In some embodiments, includes administration of a pharmaceutical composition comprising a polyphenolic phytoalexin or salt derived therefrom wherein the subject is administered a total amount of up to about 1500 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 900 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 800 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 700 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 600 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 500 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 400 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 300 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 200 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 100 mg of polyphenolic phytoalexin or salt derived therefrom per day.

In some embodiments, includes administration of a pharmaceutical composition comprising a polyphenolic phytoalexin or salt derived therefrom wherein the subject is administered a total amount of up to about 50 mg of polyphenolic phytoalexin or salt derived therefrom per day.

The invention relates to a method of treating or preventing a immuno-inflammatory disorder in a subject in need thereof by administration of a therapeutically effective amount of the pharmaceutical composition comprising polyphenolic phytoalexin or salt derived therefrom to the subject. In some embodiments, the a method of treating or preventing a immuno-inflammatory disorder in a subject in need thereof comprises the pharmaceutical composition being administered intranasally, vaginally, rectally, orally, or transdermally. In some embodiments, the method of treating or preventing a immuno-inflammatory disorder in a subject in need thereof comprises the pharmaceutical composition being administered orally. In some embodiments, the method of treating or preventing a immuno-inflammatory disorder in a subject in need thereof comprises the pharmaceutical composition being administered topically on the skin. In some embodiments, the method of treating or preventing a immuno-inflammatory disorder in a subject in need thereof comprises co-administration of the pharmaceutical composition comprising polyphenolic phytoalexin or salt derived therefrom and at least one other therapeutic agent. In some embodiments, the method of treating or preventing a immuno-inflammatory disorder in a subject in need thereof comprises administration of the pharmaceutical composition comprising polyphenolic phytoalexin or salt derived therefrom in a daily dose range of from about 0.01 mg/kg to about 500 mg/kg (or any of the amounts described herein) of polyphenolic phytoalexin or salt derived therefrom relative to the weight of the subject. In some embodiments, the method of treating or preventing a immuno-inflammatory disorder in a subject in need thereof comprises administration of a total amount of from about 1 mg to about 500 mg of polyphenolic phytoalexin or salt derived therefrom per day. In some embodiments, the method of treating or preventing a immuno-inflammatory disorder in a subject in need thereof comprises administration of up to about 40 mg of polyphenolic phytoalexin or salt derived therefrom per dose. In some embodiments of the invention, the does may be administered to a subject in need thereof in more than one dose.

In order that the invention disclosed herein may be more efficiently understood, examples are provided below. It should be understood that these examples are for illustrative purposes only and are not to be construed as limiting the invention in any manner.

### EXAMPLES

### Example 1

### Encapsulation of Resveratrol in Poly(Ethylene Oxide)-Poly(Caprolactone) Polymersomes

A poly (ethylene oxide)-β-poly(caprolactone) (PEO-b-PCL) diblock copolymer of average molecular weight from about 10 kD to about 20 kD was mixed with resveratrol (>99% purity) in weight ratios from 1:1 to 10:1 (polymer:resveratrol) in a scintillation vial. Weight percentage of PEO block in PEO-b-PCL copolymer ranged from 10 to 25%. The mixture was dissolved in a suitable organic solvent (e.g. methylene chloride, tetrahydrofuran, dimethyl sulfoxide) to produce about 0.5-2 mM solution of PEO-b-PCL copolymer. The mixture was gently shaken to produce a clear solution. About 100-500 μl of the solution was transferred on a roughened Teflon™ strip (approximately 1″×1″× 1/16″ thick) and the strip was deposited on the bottom of a glass vial with roughened side facing up. The solvent was evaporated under vacuum at ambient temperature for 24-48 hrs to obtain dried film of copolymer embedded with resveratrol.

These films were hydrated with 1-5 ml of suitable aqueous phase such as a phosphate buffered saline or sucrose solution of pH 7-7.5 and osmolality of 200-300 mOsm and the mixture was sonicated in a bath sonicator at 20-100 Hz and heating for 1-2 hrs. After the sonication was complete, hot vials were immediately vortexed for 1-2 min to form PEO-PCL polymersomes with resveratrol embedded in membrane. Polymersomes were then extruded through a polycarbonate membrane of desired pore size for about 5-15 passes using a LiposoFast™ extruder to obtain a desired average diameter of the polymersomes from about 50 nm to about 300 nm. Polymersomes were then separated using membrane filtration. The size distribution of polymersomes was confirmed using zetasizer nano S90 (Malvern Instruments, Worcestershire, UK). Quartz cuvettes were filled with 1 ml of polymersome suspensions and were thermostatically controlled at less than ambient temperatures throughout the experiment. All DLS measurements were made at a scattering angle of 90°. Encapsulation of resveratrol in polymersome membrane was verified by recording UV-Vis absorption spectra of polymersomes using a Beckman DU 800 Spectrophotometer and confirming a clear absorption peak for resveratrol at 300-320 nm wavelength.

### Example 2

### Encapsulation of Resveratrol in Poly (Ethylene Oxide)-Polybutadiene Polymersomes

A poly (ethylene oxide)-b-poly(butadiene) (PEO-b-PBD) diblock copolymer of average molecular weight from about 3 kD-15 kD was mixed with resveratrol (>99% purity) in weight ratios from 1:1 to 10:1 (polymer:resveratrol) in a scintillation vial. Weight percentage of PEO block in PEO-b-PBD copolymer ranged from 10 to 40%. The mixture was dissolved in a suitable organic solvent (e.g. methylene chloride, tetrahydrofuran, dimethyl sulfoxide) to produce about 0.5-2 mM solution of PEO-b-PBD copolymer. The mixture was gently shaken to produce a clear solution. About 100-500 μl of the solution was transferred on a roughened Teflon™ strip and the strip was deposited on the bottom of a glass vial with roughened side facing up. The solvent was evaporated under vacuum at ambient temperature for 24-48 hrs to obtain dried film of copolymer embedded with resveratrol.

These films were hydrated with 1-5 ml of suitable aqueous phase such as a phosphate buffered saline or sucrose solution of pH 7-7.5 and osmolality of 200-300 mOsm and the mixture was sonicated in a bath sonicator at 20-100 Hz and heated for 1-2 hrs. After the sonication was complete, hot vials were immediately vortexed for 1-2 min to form PEO-PBD polymersomes with resveratrol embedded in membrane. Polymersomes were then extruded through a polycarbonate membrane of desired pore size for about 10-20 passes using a LiposoFast™ extruder to obtain a desired average diameter of the polymersomes from 50 nm-300 nm. Polymersomes were then separated using membrane filtration. The size distribution of polymersomes was confirmed using zetasizer nano S90 (Malvern Instruments, Worcestershire, UK). Quartz cuvettes were filled with 1 ml of polymersome suspensions and were thermostatically controlled at less than ambient temperatures throughout the experiment. All DLS measurements were made at a scattering angle of 90°. Encapsulation of resveratrol in polymersome membrane was verified by recording UV-Vis absorption spectra of polymersomes using a Beckman DU 800 Spectrophotometer and confirming a clear absorption peak for resveratrol at 300-320 nm wavelength.

### Example 3

### Encapsulation of Resveratrol in Poly (Ethylene Oxide)-Poly (Gamma-Methyl-Epsilon-Caprolactone) Polymersomes

A poly (ethylene oxide)-β-poly(gamma-methyl-epsilon-caprolactone) (PEO-b-PMCL) diblock copolymer of average molecular weight from about 5 kD-14 kD was mixed with resveratrol (>99% purity) in weight ratios from 1:1 to 10:1 (polymer:resveratrol) in a scintillation vial. Weight percentage of PEO block in PEO-b-PMCL copolymer ranged from 20 to 40%. The mixture was dissolved in a suitable organic solvent (e.g. methylene chloride, tetrahydrofuran, dimethyl sulfoxide) to produce about 0.5-2 mM solution of PEO-b-PMCL copolymer. The mixture was gently shaken to produce a clear solution. About 100-500 μl of the solution was transferred on a roughened Teflon™ strip and the strip was deposited on the bottom of a glass vial with roughened side facing up. The solvent was evaporated under vacuum at ambient temperature for 24-48 hrs to obtain dried film of copolymer embedded with resveratrol.

These films were hydrated with 1-5 ml of suitable aqueous phase such as a phosphate buffered saline or sucrose solution of pH 7-7.5 and osmolality of 2-0-300 mOsm and the mixture was sonicated in a bath sonicator at 20-100 Hz and 25-45° C. for 1-2 hrs. After the sonication was complete, vials were immediately vortexed for 1-2 min to form PEO-PMCL polymersomes with resveratrol embedded in membrane. Polymersomes were then extruded through a polycarbonate membrane of desired pore size for about 5-15 passes using a LiposoFast™ extruder to obtain a desired average diameter of the polymersomes from 50 nm-300 nm. Polymersomes were then separated using membrane filtration. The size distribution of polymersomes was confirmed using zetasizer nano S90 (Malvern Instruments, Worcestershire, UK). Quartz cuvettes were filled with 1 ml of polymersome suspensions and were thermostatically controlled at less than ambient throughout the experiment. All DLS measurements were made at a scattering angle of 90°. Encapsulation of resveratrol in polymersome membrane was verified by recording UV-Vis absorption spectra of polymersomes using a Beckman DU 800 Spectrophotometer and confirming a clear absorption peak for resveratrol at 300-320 nm wavelength.

Data collected during encapsulation results are compiled below:

### Resveratrol Encapsulation in PEO-PBD Polymersomes

| | | Average size | | |
|---|---|---|---|---|
| | | of Resveratrol- | Peak UV | |
| | | encapsulated | absorption | |
| Batch | | Polymersomes, | wavelength, | Peak UV |
| No. | Polymer Used | nm | nm | absorbance, A |
| 1 | OB-29 (PEO₃₀- | 101 ± 17 | 311 | +2.795 |
| | PBD₄₆) | | | |
| 2 | OB-29 (PEO₃₀- | 127 ± 39 | 309 | +2.917 |
| | PBD₄₆) | | | |
| 3 | OB-29 (PEO₃₀- | 118 ± 22 | 308 | +3.000 |
| | PBD₄₆) | | | |
| 4 | OB-18 (PEO₈₀- | 107 ± 32 | 317 | +2.481 |
| | PBD₁₂₅) | | | |
| 5 | OB-18 (PEO₈₀- | 132 ± 24 | 315 | +2.696 |
| | PBD₁₂₅) | | | |

### Resveratrol Encapsulation in PEO-PCL Polymersomes

| | | Average size | | |
|---|---|---|---|---|
| | | of Resveratrol- | Peak UV | |
| | | encapsulated | absorption | |
| Batch | | Polymersomes, | wavelength, | Peak UV |
| No. | Polymer Used | nm | nm | absorbance, A |
| 1 | PEO₄₅-PCL₁₀₅ | 249 ± 77 | 314 | +2.309 |
| 2 | PEO₄₅-PCL₁₀₅ | 221 ± 89 | 308 | +2.510 |
| 3 | PEO₄₅-PCL₁₀₅ | 175 ± 58 | 318 | +2.284 |

### Resveratrol Encapsulation in PEO-PMCL Polymersomes

| | | Average size | | |
|---|---|---|---|---|
| | | of Resveratrol- | Peak UV | |
| | | encapsulated | absorption | |
| Batch | | Polymersomes, | wavelength, | Peak UV |
| No. | Polymer Used | nm | nm | absorbance, A |
| 1 | PEO₄₃-PMCL₆₆ | 253 ± 102 | 307 | +2.195 |
| 2 | PEO₄₃-PMCL₆₆ | 216 ± 61 | 311 | +1.996 |

### Example 4

### Antimicrobial and Immunological Properties of the Resveratrol-Encapsulated Polymersomes Against Proprionibacterium acnes (Prophetic Example)

The antimicrobial and immunological properties of resveratrol compartmentalized within the polymersome nanovesicle (PNV) carrier will be characterized in the presence of P. acnes and compared to that of free resveratrol. P. acnes will be cultured in the presence of increasing doses of resveratrol or resveratrol-encapsulated PNVs with subsequent viability being determined via the colony forming unit (CFU) assay. Immunological properties of resveratrol-encapsulated PNVs will be evaluated via primary human monocyte isolation and bacterial stimulation. Human peripheral mononuclear cells will be harvested and cultured via Ficoll-Paque gradients. Non-adherent cells will be removed via washing and subsequent adherent monocytes were cultured in the absence or presence of either resveratrol or the resveratrol-encapsulated PNV and P. acnes. It is well established that P. acnes stimulates monocytes to release IL-12p40, an inflammatory cytokine Presence of IL-12p40 will be measured by an ELISA assay from the supernatant 24 hours post co-culturing of monocytes and P. acnes. Flow cytometry was further utilized to evaluate the presence of known monocyte, macrophage and dendritic cell markers (e.g. CD14, CDab, CD209, and CD68 expression). In addition, monocyte viability in the presence of resveratrol or resveratrol-encapsulated PNVs will be measured via an MTT assay to evaluate toxicity. These results will demonstrate that resveratrol-encapsulated PNVs demonstrated increased efficacy in modulating the immuno-inflammatory response derived from a microbe such as P. acnes as compared to free resveratrol. Additionally, the resveratrol-encapsulated PNVs will be equivalent to free reseveratrol in regards to any dose-dependent monocyte toxicities.

### Example 5

### Co-Encapsulation of Other Pharmaceutically-Active Molecules and Resveratrol in PNV Carriers (Prophetic Example)

The size distribution of PNVs used to encapsulated resveratrol will range from about a diameter of about 50 nm to about 50 μm. In some embodiments, resveratrol was incorporated within the membrane matrix of the polymersomes. In some embodiments, the polymersome will be co-incorporated other pharmaceutically active molecules of substantial size, including those of utility in treating P. acnes, namely benzoyl peroxide, tretinoin, and clindamycin by compartmentalizing these agents in the polymersomes' aqueous core. Standard concentrations and of active compounds; benzoyl peroxide 0.1%, tretinoin 0.05%, and clindamycin will be used used in formation and compartmentalization within PNVs. In certain embodiments, the PNVs would co-encapsulate another immuno-inflammatory agent in addition to resveratrol and the pharmaceutically active molecules. In other embodiments, the incorporation of resveratrol along with other pharmaceutical and immuno-inflammatory modulating agents of interest with PNVs was achieved using well-established formation protocols reliant upon polymersome self-assembly.

### Example 6

### Time-Release Assays and Evaluation of PNV Parameters Under Varying pH and Temperature Environments (Prophetic Example)

Time-release assays will be performed to demonstrate that PNVs released resveratrol in a time dependent manner that was analogous to the release of other water-insoluble molecules that were incorporated within the polymersome membrane. The controlled release of resveratrol from polymeric carriers is critical to increasing active drug localization to the skin, but also in minimizing side effects and irritation of topical drug preparations. In some embodiments, the exact chemical composition of the biodegradable polymers constituting the membranes of the PNVs will be altered in order to tailor the release of resveratrol over various time periods ranging from minutes to hours when exposed to different environments, including both pH and temperature variables. Shorter release times will be tested by utilizing biodegradable polymersomes comprised of diblock copolymers of poly(ethylene oxide) and either poly(D-lactic acid), poly(L-lactic acide), poly(glycolic acid), and/or poly(lactic-co-glycolic acid). Longer release times will be tested by utilizing biodegradable polymersomes comprised of diblock copolymers of poly(ethylene oxide) and either poly(ε-caprolactone) or poly(γ-methyl ε-caprolactone). Release of resveratrol from poly(ethylene oxide)-block-poly(butadiene)-based polymersomes will be measured by the diffusion of resveratrol across intact vesicle membranes and will be further slowed by varying the weight fraction of the block copolymer that formed the polymersome membrane such as to increase its size. As such, altering diblock copolymer composition and total molecular weight may be two effective methods to vary the rate and duration of resveratrol release from polymeric carriers such as to maximize efficacy while minimizing side-effect profiles from either free reseveratrol or resveratrol co-encapsulated with other pharmaceutically active molecules or immuno-inflammatory modulating agents.

1. A polymeric carrier comprising:
i) a plurality of copolymers; and
ii) at least one polyphenolic phytoalexin compound that is dispersed within the polymer matrix (or within the aqueous core or a polymer vesicle/polymersome); or at least one polyphenolic phytoalexin compound and at least one pharmaceutically active agent that are dispersed within the polymer matrix or within the aqueous core of a polymer vesicle/polymersome.

2. The polymer carrier of claim 1 where the carrier is comprised of at least one biocompatible polymer.

3. The polymer carrier of claim 1 where the carrier is a comprised of at least one biodegradable polymer.

4. The polymer carrier of claim 2 where the carrier is a polymersome comprised of a biocompatible copolymer of poly(ethylene oxide) or poly(ethylene glycol) and either poly(butadiene), poly(ethyl-ethylene), or poly(methyl-methacrylate).

5. The polymer carrier of claim 3 where the carrier is a polymersome comprised of a biodegradable copolymer of poly(ethylene oxide) or poly(ethylene glycol) and either poly(ε-caprolactone), poly(γ-methyl ε-caprolactone), poly(L-lactic acid), poly(D-lactic acid), poly(glycolic acid), poly(L-lactic-co-glycolic acid), or poly(D-lactic-co-glycolic acid).

6. The polymer carrier of claim 3 where the carrier is a polymersome comprised of a biodegradable copolymer of poly(ethylene oxide) or poly(ethylene glycol) and either a poly(peptide), poly(saccharide), or poly(nucleic acid).

7. The polymer carrier of claim 5 where the polymersome-forming biodegradable polymer is a block copolymer of poly(ethylene oxide) and poly(ε-caprolactone), the polyethylene oxide having a number average molecular weight from about 1.5 to about 3.8 kD, the block copolymer having a fraction of polyethylene oxide from about 11 to about 20 percent by weight.

8. The polymer carrier of claim 5 where the polymersome-forming biodegradable polymer is a block copolymer of poly(ethylene oxide) and poly(γ-methyl-ε caprolactone), the polyethylene oxide having a number average molecular weight from about 1.5 to about 3.8 kD, the block copolymer having a fraction of polyethylene oxide from about 17 to about 28 percent by weight.

9. The polymer carrier of claim 5 where the polymersome-forming biodegradable polymer is a block copolymer in which at least one block is poly(ethylene oxide) and one block is poly
(ε-caprolcatone), the polyethylene oxide having a number average molecular weight from about 1.5 to about 3.8 kD, the block copolymer having a fraction of polyethylene oxide of from about 10 to about 30 percent by weight.

10. The polymer carrier of claim 5 where the polymersome-forming biodegradable polymer is a block copolymer in which at least one block is poly(ethylene oxide) and one block is poly (γ-methyl ε-caprolcatone), the polyethylene oxide having a number average molecular weight from about 1.5 to about 3.8 kD, the block copolymer having a fraction of polyethylene oxide of from about 10 to about 30 percent by weight.

11. The polymer carrier of claim 1 where the carrier is a comprised of block copolymers and at least one polymer block is poly(ethylene oxide) or poly(ethylene glycol), wherein the weight fraction of poly(ethylene oxide) or poly(ethylene glycol) is from about 30 to about 507 percent by weight.

12. The polymer carrier of claim 1 where the carrier is a comprised of block copolymers and at least one polymer block is poly(ε-caprolactone), wherein the weight fraction of poly(ε-caprolactone) is from about 50 to 70 percent by weight.

13. The polymer carrier of claim 1 where the carrier is a comprised of block copolymers and at least one polymer block is poly(γ-methyl ε-caprolactone), wherein the weight fraction of poly(γ-methyl ε-caprolactone) is from about 50 to about 70 percent by weight.

14. The polymer carrier of claim 7 wherein the polymersome-forming diblock copolymer has a number average molecular weight of poly(ε-caprolactone) that is from about 9 to about 23 kD.

15. The polymer carrier of claim 8 wherein the polymersome-forming diblock copolymer has a number average molecular weight of poly(γ-methyl ε-caprolactone) that is from about 4 to 10 kD.

16.-17. (canceled)

18. The polymer carrier of claim 7 wherein the polymersome-forming diblock copolymer has a molecular weight of the poly(ethylene oxide) that is about 2 kD and a molecular weight of poly(ε-caprolactone) that is about 12 kD.

19. The polymer carrier of claim 8 wherein the polymersome-forming diblock copolymer has a molecular weight of poly(ethylene oxide) that is 1.8-1.9 kD and a molecular weight of poly(γ-methyl ε-caprolactone) that is about 8-9.5 kD.

20. The polymer carrier of claim 4 that is a polymersome in which resveratrol is compartmentalized within its aqueous interior, is compartmentalized within the within the hydrophobic polymersome membrane, or is covalently linked to the polymersome's hydrophilic surface.

21.-22. (canceled)

23. The polymersome of claim 20 where the compartmentalized resveratrol is combined with another pharmaceutically active agent.

24.-27. (canceled)

28. The polymersome of claim 20 where the compartmentalized resveratrol is of therapeutic value to the treatment of immuno-inflammatory disorders or to the treatment of acne vulgaris.

29.-32. (canceled)

33. A method of treating an immunoinflammatory disorder in an individual comprising administering to the individual:
a) an effective amount of a composition comprising a polyphenolic phytoalexin compound or derivative thereof encapsulated within a polymeric carrier, or
b) an effective amount of a composition comprising a polyphenolic phytoalexin compound or derivative thereof and a pharmaceutically active agent that are co-encapsulated within and a polymeric carrier, and
c) an effective amount of a composition of at least one other anti-immunoinflammatory agent, wherein the anti-immunoinflammatory agent is chosen from an antibiotic, a chemotherapeutic agent, vitamin A or a derivative thereof, laser therapy, ultraviolet therapy, retinoic acid receptor and a retinoid X receptor modulator, and benzoyl peroxide.

34. The method according to claim 33, wherein the diameter of the polymeric carrier ranges from 50 nm-300 nm in size.

35. The method according to claim 33, wherein the polymeric carrier is a polymersome.

36. The method according to claim 33, wherein the polymeric carrier is a polymersome that is comprised of a plurality of copolymers.

37. The method according to claim 36, wherein the polymersome is comprised of a biocompatible or biodegradable polymer.

38. The method according to claim 36, wherein the polymersome is comprised of block copolymers of poly(ethylene oxide) and poly(ε-caprolactone).

39. The method according to claim 36, wherein the polymersome is a comprised of block copolymers or poly(ethylene oxide) and poly(γ-methyl ε-caprolactone).

40. The method according to claim 33, wherein the polyphenolic phytoalexin compound or derivative thereof is resveratrol (3,5,4′-trihydroxystilbene).

41. The method according to claim 33, wherein the pharmaceutically active agent is a protein, peptide, saccharide, nucleoside, inorganic compound, or organic compound.

42. The method according to claim 33, wherein the anti-immunoinflammatory agent is an antibiotic.

43. The method according to claim 42, wherein the antibiotic is a tetracycline, macrolide, fluoroquinolone, lincosamide, aminoglycoside, sulfonamide, sulfapyradine, chloramphenicol, vancomycin, metronidazole, oxazolidinone.

44. The method according to claim 33, wherein the anti-immunoinflammatory agent is a chemotherapeutic agent.

45. The method according to claim 44, wherein the chemotherapeutic agent is an antimetabolite, alkylating agent, proteasome inhibitor, tyrosine kinase inhibitor, anthracycline, vinca alkaloid, platinum based agent, and topoisomerase inhibitor.

46. The method according to claim 33, wherein Vitamin A and derivatives thereof include all-trans-retinoic acid, 13-cis-retinoic acid, retinal, and retinyl esters.

47. The method according to claim 33, wherein laser therapy includes argon, frequency-doubled Nd:YAG/KTP, pulsed dye, ruby, alexandrite, diode, Nd:Yag, carbon dioxide, erbium:YAG.

48. The method according to claim 33, wherein ultraviolet therapy includes irradiations with broadband UVB (290-320 nm), narrowband UVB (311-313 nm), 308 nm excimer laser, UVA1 (340-400 nm), UVA plus psoralens (PUVA), and extracorporeal photochemotherapy.

49. The method according to claim 33, wherein the anti-immunoinflammatory agent is a retinoic acid receptor, retinoid X receptor modulator, or benzoyl peroxide.

50. (canceled)

51. The method according to claim 33, wherein the immunoinflammatory disorder is acne vulgaris.

52. The method according to claim 33, wherein the polyphenolic phytoalexin compound or derivative thereof is administered topically, transdermally, per rectum, per the vagina, intravenously, orally, subcutaneously, intraperitoneally, intrathecally, intramuscularly, or via inhalation.
